(19) United States
(12) Patent Application Publication
(10) Pub. No.: US 2026/0315657 A1
(43) Pub. Date: Oct. 15, 2026

(54) MESHED UMBILICAL CORD TISSUE GRAFTS

(71) Applicant: MiMedx Group, Inc., Marietta, GA (US)

(72) Inventors: Adam McQueen, Marietta, GA (US); Rick LeVaughn, Marietta, GA (US); Thomas J. Koob, Marietta, GA (US); Michelle Massee, Marietta, GA (US)

(21) Appl. No.: 19/381,526

(22) Filed: Nov. 6, 2025

Related U.S. Application Data
(63) Continuation of application No. 17/350,103, filed on Jun. 17, 2021, now abandoned.
(60) Provisional application No. 63/041,598, filed on Jun. 19, 2020.

(51) Int. Cl. A61L 27/36

(52) U.S. Cl. CPC A61L 27/3695; A61L 27/3604; A61L 2400/06; A61L 2430/34

(57) ABSTRACT

A meshed, dehydrated, umbilical tissue allograft that can be used in the treatment of wounds. Specifically, the meshed allograft has the property of being able to be expanded to cover an irregularly shaped wound, therefore reducing the need to apply multiple, uniform sized grafts to a single wound site. The meshed, dehydrated umbilical tissue allograft is sourced from a human donor, and is then processed to remove any potential contaminants or microbes prior to applying a specific mesh pattern to the tissue. The meshed, dehydrated, umbilical tissue graft is reconstituted prior to applying to the subject, and can then be configured to optimally cover the shape of the wound site.

### CROSS REFERENCE TO RELATED APPLICATIONS

This application is a continuation of U.S. application Ser. No. 17/350,103, filed Jun. 17, 2021, which claims the benefit of and priority to U.S. Provisional Application No. 63/041,598, filed Jun. 19, 2020, the content of each of which is hereby incorporated by reference in its entirety.

### FIELD OF THE INVENTION

The invention relates to expandable, dehydrated, meshed umbilical cord tissue allografts that can be rehydrated and expanded to cover a variety of wounds having an irregular shape.

### BACKGROUND

Human placental membrane (e.g. amniotic membrane or tissue) has been used for various types of reconstructive surgical procedures since the early 1900s. The membrane serves as a substrate material, more commonly referred to as a biological dressing or patch graft. Such membrane has also been used widely for ophthalmic procedures in the United States and in countries in the southern hemisphere. Typically, such membrane is either frozen or dried for preservation and storage until needed for surgery.

Such placental tissue is typically harvested after an elective Cesarean surgery. The expelled placenta and associated tissues are composed of the umbilical cord, the placental disk, and the amniotic sac (or membrane). The amniotic sac, commonly referred to as the amniotic membrane, has two primary layers of tissue, amnion and chorion. Amnion tissue is innermost layer of the amniotic sac and in direct contact with the amniotic fluid. The amniotic sac contains the amniotic fluid and protects the fetal environment. Histological evaluation of this tissue indicates that the membrane layers of the amniotic membrane consist of epithelium cells, thin reticular fibers (basement membrane), a thick compact layer, and fibroblast layer. The fibrous layer of amnion (i.e., the basement membrane) contains cell anchoring collagen types IV, V, and VII. The chorion is also considered as part of the fetal membrane; however, the amniotic layer and chorion layer form a continuous layer in vivo, but can easily be separated upon removal from the body and upon subsequent dissection of the tissue.

Amniotic membrane allografts have been successfully used as a biologic therapy to promote soft tissue healing; however, the umbilical cord, another placental-derived tissue, has also recently garnered interest because of its unique composition and its similar placental tissue origin.

Amniotic membrane has been used for various types of reconstructive surgical procedures since the early 1900s. The membrane serves as a as a biological dressing or patch graft. Such a membrane has also been used widely for ophthalmic procedures. Typically, such membrane is either frozen or dried for preservation and storage until needed for surgery.

The umbilical cord contains Wharton's jelly, a gelatinous substance made largely from mucopolysaccharides which protects the blood vessels inside. It contains one vein, which carries oxygenated, nutrient-rich blood to the fetus, and two arteries that carry deoxygenated, nutrient-depleted blood away. Occasionally, only two vessels (one vein and one artery) are present in the umbilical cord. This is sometimes related to fetal abnormalities, but it may also occur without accompanying problems.

Human umbilical cord tissue contains collagen I, hyaluronic acid, laminin, and fibronectin. Additionally, at least 504 proteins that consist of growth factors and cytokines, inflammatory modulators, chemokines, proteases and inhibitors, adhesion molecules, signaling receptors, membrane-bound proteins, and other soluble regulators have been observed to date in the tissue. Cell-based assays have demonstrated an increase in adipose-derived stem cell and mesenchymal stem cell proliferation, fibroblast migration and endothelial progenitor cell vessel formation in a dose-dependent manner after treatment with dehydrated human umbilical tissue.

However, the umbilical cord is generally limited by its size and dimension, and not amenable to use to treat larger or unusually shaped wounds. The subject matter described herein addresses the shortcomings.

### SUMMARY OF THE INVENTION

In certain embodiments, the subject matter described herein is directed toward a meshed, dehydrated and sterile, umbilical tissue graft, and particularly human allograft, that is expandable to cover the shape of a wound site that is extensive and often irregularly shaped.

In one embodiment, this invention provides for a dehydrated and sterile, umbilical tissue allograft comprising a specific pattern of cuts that permit expansion of the graft without significantly compromising its structural integrity. In an embodiment, these cuts or incisions have been incorporated into the graft after the tissue has been decontaminated and dehydrated. Based on the particular embodiment, the cuts can be applied by a commercially available meshing device, a laser cutting device, or a cutting template. Additionally, the dehydration step may be carried out by well-known techniques in the art, such as air drying, chemical drying, or lyophilization of the tissue. Additionally, certain embodiments comprise a mesh pattern which enhances the expansion capabilities of the dehydrated umbilical tissue graft. In embodiments, multiple, orthogonal, engineered, and pre-determined mesh patterns may be applied to a single graft.

After harvesting, the umbilical cord is treated in a number of steps to provide the products described herein. For example, the cord is separated from the amnion, chorion, and placental disc tissue (the other tissues may be retained for other purposes). All components are sourced from a single donor. The umbilical cord is subject to a specific process in which it is rinsed in a salt solution, and then rinsed again in an antibiotic solution, and then a final rinse in yet another salt solution designed to remove residual antibiotic solution from the tissue.

In an embodiment, the umbilical cord tissue is initially cleaned in a hyperisotonic solution wherein the hyperisotonic solution comprises NaCl concentration in a range of from about 30% to about 10%.

The vein and arteries are removed and the remaining umbilical tissue is gently cleansed and minimally manipulated to preserve inherent growth factors and proteins in the tissue. Notable growth factors in the umbilical cord include transforming growth factor beta (TGF-β), basic fibroblast growth factor (bFGF), platelet derived growth factors (PDGF AA & BB), and vascular endothelial growth factor (VEGF)14,15, which are known to regulate wound healing.

After the decontamination steps have been carried out, the umbilical cord tissue is then subjected to a drying process, which may involve any type of commercially acceptable process known in the art, including, but not limited to air drying, chemical drying or lyophilization of the umbilical cord tissue.

After the umbilical cord tissue has been dried, a specific cut pattern is applied which gives the tissue a meshed appearance. Based on the desired embodiment, the cut pattern can be varied, and may be applied through the use of commercially available apparatuses, such as a meshing tool, a laser cutting tool or a cutting template.

The finished product is packaged in a sterile container and is reconstituted with an acceptable excipient by the end user before the graft is applied to the subject's wound site. Alternatively, the graft can be applied directly to the wound site and can be reconstituted with a combination of an excipient and the patient's own bodily fluids that may be present in the wound site.

In one embodiment, there is provided a method for forming an expandable umbilical cord tissue graft having structural integrity which method comprises a) obtaining a dehydrated umbilical cord tissue graft, b) placing a set of cuts or incisions into said graft to permit expansion of the graft; and c) sterilizing said graft.

### BRIEF DESCRIPTION OF THE FIGURES

Further features and benefits of the present invention will be apparent from a detailed description of preferred embodiments thereof taken in conjunction with the following drawings, wherein similar elements are referred to with similar reference numbers, and wherein:

FIG. 1 illustrates a slit mesh pattern prototype very similar in pattern to the slits achieved by a traditional skin graft mesher, but the slit lengths and spacing are set to achieve peak expansion of the meshed graft. The expanded graft has a 2.5 mm thick border on the long edge of the graft and 1.5 mm thick strands of tissue surrounding large holes where incisions are made. The tissue expands along the short axis of the graft. Both laser and die cut methods of manufacturing can be utilized to incorporate this pattern.

FIG. 2 illustrates a zig zag mesh pattern. When expanded, this mesh pattern creates small holes with strips of tissue covering each small hole. This mesh pattern results in tissue strands that are diagonally positioned and very close together allowing the graft to maintain its structural integrity during handling and suturing.

FIG. 3 illustrates a spiderweb mesh pattern created by positioning the slit design at different angles from the center of the graft to create a hexagonal effect.

FIG. 4 illustrates an “evil eye” mesh pattern comprising a dense series of straight and slanted slits.

FIG. 5 illustrates a modified version of the evil eye mesh pattern designed to create thicker strands of tissue compared to the original evil eye mesh pattern. The number of slits is reduced significantly in relation to this mesh pattern.

FIG. 6 illustrates another evil eye mesh pattern designed to create thicker strands of tissue, which allows for a greater degree of expansion. The number of slits is reduced slightly in comparison with the other two evil eye mesh patterns.

FIG. 7 illustrates the dehydrated, meshed, umbilical cord graft in its native state, prior to reconstitution or expansion.

FIG. 8 illustrates the reconstituted, meshed umbilical cord graft after a desired excipient has been applied, and force has been applied, causing the graft to expand.

### DETAILED DESCRIPTION OF THE INVENTION

One challenge that has been encountered in the field is that umbilical tissue grafts (as well as all tissue grafts in general) are of a uniform shape and size, even though wounds tend to be irregularly shaped. Umbilical cord allograft sizes are constrained by the dimensions of the source cord tissue itself; therefore, larger wounds or injury sites may require multiple grafts, which may be cost prohibitive, or preclude the use of an umbilical tissue graft entirely.

Accordingly, there is a need in the marketplace for an efficient manner of applying a graft over an extended and often irregularly shaped wound. One potential solution involves expanding the umbilical cord tissue via a meshing process that allows for the expansion of the tissue and an accompanying increase in the coverage area. The creation of a graft that can be spread to fit an irregular wound shape would enhance efficiency and potentially reduce the cost of treatment.

Moreover, tissue grafts prepared from umbilical cord are naturally limited in size in at least one axis, in a way that grafts made from other placental tissue (like amnion and/or chorion), or from skin, are not. The average circumference of an umbilical cord at birth is approximately 4.5 cm. In cases where large wounds necessitate similarly-sized grafts, practitioners have not looked to use umbilical grafts, because grafts from other sources could provide a single, larger graft without requiring expansion. Consequently, practitioners have not looked to umbilical cord grafts as a matter of convenience, because grafts from other sources have been more suitable for larger grafts. The present inventors have found that umbilical cord grafts may be made to expand without substantially reducing their structural integrity, so as to make them suitable for more universal use. Umbilical tissue is a limited resource. Advantageously, the grafts and methods described herein provide for more efficient use of available tissue in light of its relative scarcity.

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the manufacture, practice or testing of the present invention, the preferred methods and materials are now described. All patents and publications mentioned herein are incorporated by reference to disclose and describe the methods and/or materials in connection with which the publications are cited.

Each embodiment disclosed herein is contemplated as being applicable to each of the other disclosed embodiments. All combinations and sub-combinations of the various elements described herein are within the scope of the embodiments.

It is understood that where a parameter range is provided, all integers and ranges within that range, and tenths and hundredths thereof, are also provided by the embodiments. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%, as well as, for example, 6-9%, 5.1%-9.9%, and 5.01%-9.99%. This also applies to ratios. For example, a recited ratio range of “1:100 to 200:1” includes ratios such as 1:50, 1:1, and 100:1, along with ranges such as 1:100 to 1:1, 1:50 to 50:1, and 1:1 to 200:1.

As used herein, “about” in the context of a numerical value or range means within ±1%, ±5%, or 10% of the numerical value or range recited or claimed.

The invention illustratively disclosed herein suitably may be practiced in the absence of any element which is not specifically disclosed herein.

It must be noted that as used herein and in the appended claims, the singular forms “a”, “an”, and “the” include plural referents unless the context clearly dictates otherwise.

### Definitions

As used herein the following terms have the following meanings.

“Placental tissue” or “placenta” means the umbilical cord, the placental disk, and the amniotic sac.

“Comprising” or “comprises” is intended to mean that the compositions, for example media, and methods include the recited elements, but not excluding others. “Consisting essentially of” when used to define methods, shall mean excluding other elements of any essential significance to the combination for the stated purpose. “Consisting of” shall mean excluding additional substantial method steps. Embodiments defined by each of these transition terms are within the scope of this invention.

“Dehydrated” means that the tissue has had substantially all of its water removed, (i.e. greater than 90%, greater than 95%, greater than 99%, or 100% of its water removed).

“Optional” or “optionally” means that the subsequently described event or circumstance can or cannot occur, and that the description includes instances where the event or circumstance occurs and instances where it does not.

The term “subject” as used herein is any vertebrate organism including but not limited to mammalian subjects such as humans, farm animals, domesticated pets and the like. The term “patient” may be used interchangeably with “subject.”

The term “meshed” refers to umbilical cord grafts which have had a plurality of cuts made in an engineered pattern, through the entirety of the thickness of the graft. These cuts form holes when the graft is expanded along at least one axis. The cuts may be of varying size, and may vary in distance from one another, or in their orientation relative to each other.

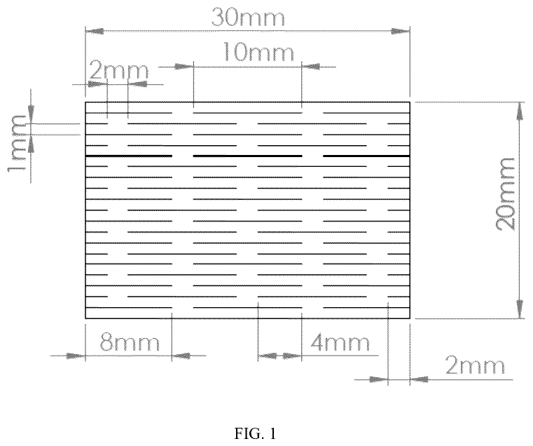

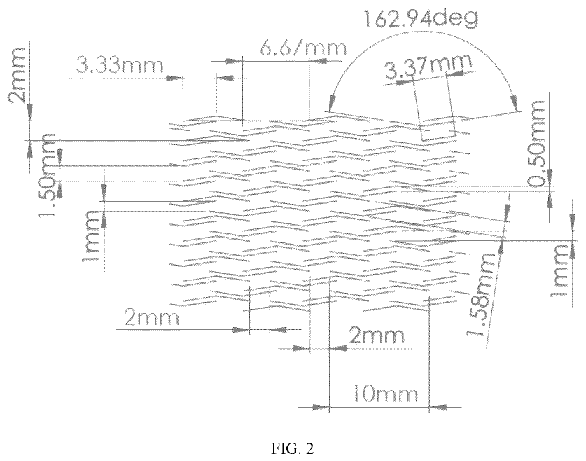

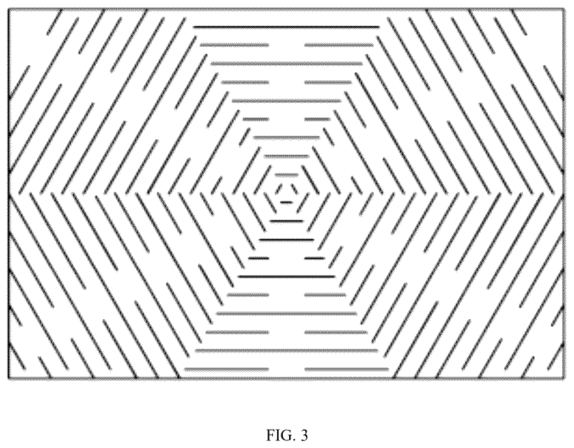

The term “engineered pattern” refers to a specific arrangement of cuts made in an umbilical cord graft that are not random. For example, an engineered pattern may consist of a plurality of identically-sized cuts, all parallel to each other (as in FIG. 1). Alternatively, the engineered pattern may comprise cuts which are at an angle relative to some other cuts (as in FIG. 2), or cuts which are at three or more distinct angles relative to each other (as in, e.g., FIGS. 3-6).

The term “treat,” with respect to a wound, means to reduce the amount of time the wound would have taken to heal in the absence of any type of medical intervention.

The term “cuts” refers to any of a number of incisions in the mesh including but not limited to line cuts (or “slits”), hole punches which can be circular, ovular, rectangular, rhomboid, or irregularly-shaped holes, or combinations thereof. A “slit” is another name for a linear cut that does not remove any tissue from the graft, as would a hole punch.

The term “expandable” means the ability of the tissue graft to expand by at least 10% of its original size, or by at least 10% over at least one axis as compared to its natural shape when acted upon by an outside force, thereby excluding expansion solely due to rehydration. An “expanded” tissue graft is one that has been expanded by at least 10%, and is maintained in an expanded form after the external force is removed.

Preferably, the graft can be expanded by at least 100% of its original size, or by 100% over at least one axis. Ideally, the graft can be expanded by at least 200% of its original size, or by at least 200% over at least one axis. In an embodiment, the graft is expanded by stretching the graft. Stretching, in this context, does not infer elastic properties. The stretching and expansion through the application of an external force over one axis is at all times to be differentiated from the slight increase in volume that the graft experiences when it is rehydrated or reconstituted prior to, or during application of, the graft to the wound site.

The term “tensile strength” means the amount of force that a graft can withstand while being stretched or expanded before failing or breaking.

The term “expansion ratio” refers to the surface area that an unexpanded meshed graft (i.e., in its natural shape) can cover compared to the same meshed graft in its expanded form. For example, a meshed graft having an expansion ratio of 1:3 will cover 3 times as much surface area in its expanded state. This is equivalent to an increase of 200% in surface area coverage.

### Detailed Description

An embodiment is a meshed, expandable, and sterile umbilical tissue allograft.

In an embodiment, said allograft comprises pre-determined cuts.

In an embodiment, the cuts are in an engineered pattern.

In an embodiment, all of said cuts are parallel to one another.

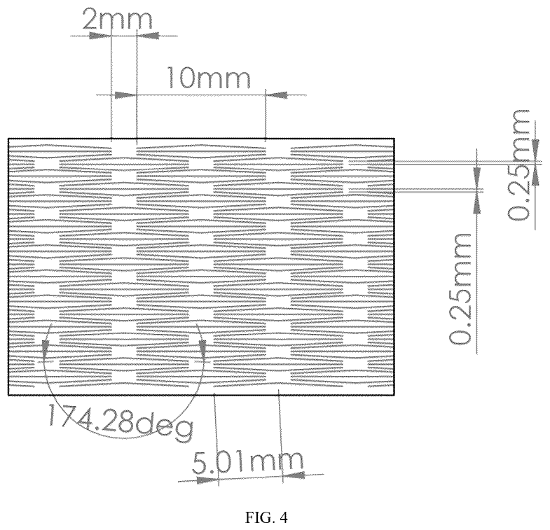

In an embodiment, said cuts are 0.1-20 mm in length. It is understood that not all cuts in a graft need to be the same size and/or shape, due to either the pattern of cuts chosen (as in, for example, FIG. 4) or due to cuts at the edge of an allograft (as in, for example, FIG. 1). In an embodiment, said cuts are 0.2-10 mm, 0.4-6 mm, or 0.5-5 mm in length. In an embodiment, said cuts are greater than, less than, or about 0.2, 0.3, 0.4, 0.5, 0.6, 0.7, 0.8, 0.9, 1, 1.5, 2, 2.5, 3, 3.5, 4, 4.5, 5, 6.5, 7, 7.5, 8, 8.5, 9, 9.5, 10, 11, 12, 13, 14, 15, 16, 17, 18, or 19 mm in length.

In an embodiment, the distance between two adjacent cuts is between 0.1-10 mm. It is understood that “adjacent” refers to, in various embodiments, to the distance between ends of two cuts (an end-to-end distance of 2 mm in FIG. 1) or to the distance between two cuts which run parallel to each other but are not on the same line (a side-to-side distance of 1 mm in FIG. 1).

In an embodiment, the ratio of cut length to the distance between two adjacent cuts is from 1:100 to 200:1. In an embodiment, the ratio is from 1:50 to 100:1. In an embodiment, the ratio is from 1:20 to 25:1. In an embodiment, the ratio is from 1:10 to 10:1. In an embodiment, the ratio is greater than, less than, or about 1:50, 1:25, 1:20, 1:10, 1:5, 1:2, 1:1, 2:1, 5:1, 10:1, 20:1, 25:1, 50:1, 100:1, or 150:1.

In an embodiment, a first portion of said cuts are parallel to one another, a second portion of said cuts are parallel to one another, and the first portion and the second portion are oriented at an angle of 1°-180° relative to each other. An example of such a pattern is shown in FIG. 2. In an embodiment, said angle is greater than, less than, or about 1°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, or 175°.

In an embodiment, the allograft further comprises a third portion of cuts which is not parallel to the first portion or to the second portion. An example of such a pattern is shown in FIG. 3.

In an embodiment, the allograft comprises no more than five portions of cuts, wherein each of said no more than five portions of cuts are oriented at an angle of 1°-180° relative to each of said other portions.

In an embodiment, said cuts comprise no more than 20%, 15%, 10%, 9%, 8%, 7%, 6%, 5%, 4%, 3%, 2%, 1%, 0.5%, or 0.1% of the surface area of the allograft.

In an embodiment, said cuts are slits.

In an embodiment, the allograft may be expanded by at least 10%-400% along one axis by application of a force that is less than its tensile strength. In an embodiment, the allograft may be expanded so as to increase its surface area by at least 10%-400% by application of a force that is less than its tensile strength. In embodiments, said expansion or increase is at least or about 10, 20, 30, 40, 50, 60, 70, 80, 90, 100, 120, 140, 160, 180, 200, 220, 240, 260, 280, 300, 320, 340, 360, 380, or 400%.

In an embodiment, the umbilical tissue allograft is dehydrated. In an embodiment, the umbilical tissue allograft is lyophilized.

In an embodiment, the allograft is contained within a sealed pouch. In an embodiment, the sealed pouch is deoxygenated.

An embodiment of the invention is also a method of covering a wound comprising, contacting a wound with a sterile umbilical tissue allograft as described herein, wherein said contacting comprises expanding said sterile umbilical tissue allograft by at least 10%-400% along one axis to cover said wound.

An embodiment of the invention is also a method of treating a subject in need thereof using the umbilical tissue allograft as described herein, said method comprising
i) expanding said umbilical tissue allograft by at least 10%-400% along one axis, or expanding said umbilical tissue allograft so as to increase its surface area by at least 10%-400%; and
ii) applying the expanded umbilical tissue allograft to the subject.

### Methods of Manufacture

In embodiments, the subject matter described herein is directed to a method of preparing a meshed, expandable, sterile umbilical tissue graft, comprising: contacting an umbilical tissue with a cutting tool to introduce pre-determined cuts in the umbilical tissue to prepare the meshed, expandable, sterile umbilical tissue graft. In embodiments, the pre-determined cuts are in an engineered pattern. In embodiments, the cutting tool is selected from the group consisting of a laser, a cutting template, a die cutter, a mesher, and the like. In certain embodiments, the pre-determined cuts are in an engineered pattern, wherein the cutting tool is not a mesher. In certain embodiments, the umbilical tissue is dehydrated prior to the contacting of the cutting tool. In certain embodiments, the umbilical tissue is dried prior to the contacting of the cutting tool. In certain embodiments, the method further comprises sterilizing and packaging the meshed, expandable, sterile umbilical tissue graft in a package, such as a pouch. In certain embodiments, the subject matter described herein is a meshed, expandable, sterile umbilical tissue graft prepared by any of the methods described above.

### Initial Tissue Collection

The recovery of the umbilical tissue originates in a hospital, where it is collected during a Cesarean section birth. The donor, referring to the mother who is about to give birth, voluntarily submits to a comprehensive screening process designed to provide the safest tissue possible for transplantation. The screening process preferably tests for antibodies to the human immunodeficiency virus type 1 and type 2 (anti-HIV-1 and anti-HIV-2), hepatitis B surface antigens (HBsAg), antibodies to the hepatitis C virus (anti-HCV), antibodies to the human T-lymphotropic virus type I and type H (anti-HTLV-I and anti-HTLV-II), CMV, and syphilis, using conventional serological tests. The above list of tests is exemplary only, as more, fewer, or different tests may be desired or necessary over time or based upon the intended use of the grafts, as will be appreciated by those skilled in the art.

Based upon a review of the donor's information and screening test results, the donor will either be deemed acceptable or not. In addition, at the time of delivery, cultures are taken to determine the presence of, for example, Clostridium or Streptococcus. If the donor's information, screening tests, and the delivery cultures are all negative (i.e., do not indicate any risks or indicate acceptable level of risk), the donor is approved and the tissue specimen is designated as initially eligible for further processing and evaluation.

Human placentas that meet the above selection criteria are preferably individually bagged in a saline solution in a sterile shipment bag and stored in a container of wet ice for shipment to a processing location or laboratory for further processing.

### Material Check-In and Evaluation

Upon arrival at the processing center or laboratory, the shipment is opened and verified that the sterile shipment bag/container is still sealed and intact, that ice or other coolant is present and that the contents are cool, that the appropriate donor paperwork is present, and that the donor number on the paperwork matches the number on the sterile shipment bag containing the tissue. The sterile shipment bag containing the tissue is then stored in a refrigerator until ready for further processing. All appropriate forms are completed and chain of custody and handling logs are also completed.

### Gross Tissue Processing Step

When the tissue is ready to be processed further, the sterile supplies necessary for processing the placenta tissue further are assembled in a staging area in a controlled environment and are prepared for introduction into a critical environment. If the critical environment is a manufacturing hood, the sterile supplies are opened and placed into the hood using conventional sterile technique. If the critical environment is a clean room, the sterile supplies are opened and placed on a cart covered by a sterile drape. All the work surfaces are covered by a piece of sterile drape using conventional sterile techniques, and the sterile supplies and the processing equipment are placed on to the sterile drape, again using conventional sterile technique.

If the placenta tissue is collected prior to the completion or obtaining of results from the screening tests and delivery cultures, such tissue is labeled and kept in quarantine. The tissue is approved for further processing only after the required screening assessments and delivery cultures, which declare the tissue safe for handling and use, are satisfied.

Processing equipment is decontaminated according to conventional and industry-approved decontamination procedures and then introduced into the critical environment. The equipment is strategically placed within the critical environment to minimize the chance for the equipment to come in proximity to or be inadvertently contaminated by the tissue specimen.

Next, the placenta is removed from the sterile shipment bag and transferred aseptically to a sterile processing basin within the critical environment. The sterile basin contains, preferably, 18% NaCl (hyperisotonic saline) solution that is at room or near room temperature. The umbilical cord will then be held up with one hand so that the base of the umbilical cord can be located where it connects with the placental disc. Sterile scissors are then used to cut the umbilical cord away from the placental disc tissue. The umbilical cord is then placed in a sterile bowl for further processing.

Next, if the umbilical cord is deemed acceptable for further processing, the umbilical cord is inspected and the large blood vessel is located at one end of the cord tissue. The materials and equipment used in this procedure include the processing tray, 18% saline solution, and two sterile Nalgene jars. The end of the cord can be trimmed if the vessels are not immediately visible or exposed.

With the umbilical cord in the processing tray, a large rod is inserted into the large vessel and inserted as far in as possible, without puncturing the wall of the vessel. Using a pair of scissors and/or a sterile scalpel, the vessel is then cut along the slot in the rod to open the cord and expose the interior of the large vessel. Using a small curved forceps, the lining of the vessel is then removed.

Next, the small vessel is located at the end of the cord. A small rod is inserted into this vessel, again, taking care not to puncture the wall of the small vessel. Using a pair of scissors and/or sterile scalpel, the vessel is then cut down its entire length. The pieces of the vessel that have been exposed should be removed. This process is then repeated with the other small vessel. After the vessels have been exposed and removed, the umbilical cord is then opened up. After cutting the umbilical cord open, the cord is rinsed in a bowl of 18% hyperisotonic saline solution to remove any large blood clots that may be present in the tissue.

### Chemical Decontamination Step

The retained umbilical cord tissue is then placed into a sterile Nalgene jar for the next step of chemical decontamination. Any undesired umbilical cord tissue components are discarded in an appropriate biohazard container.

Next, the Nalgene jar is aseptically filled with 18% saline solution and sealed (or closed with a top). The jar is then placed on a rocker platform and agitated for between 30 and 90 minutes, which further cleans the umbilical tissue of any residual contaminants.

If the rocker platform was not in the critical environment (e.g., the manufacturing hood), the Nalgene jar is returned to the critical/sterile environment and opened. Using sterile forceps, the umbilical cord tissue is gently removed from the Nalgene jar containing the 18% hyperisotonic saline solution and placed into an empty Nalgene jar. This empty Nalgene jar with the tissue is then aseptically filled with a pre-mixed antibiotic solution. Preferably, the premixed antibiotic solution is comprised of a cocktail of antibiotics, such as Streptomycin Sulfate and Gentamicin Sulfate. Other antibiotics, such as Polymixin B Sulfate and Bacitracin, or similar antibiotics now available or available in the future, are also suitable. Additionally, it is preferred that the antibiotic solution be at room temperature when added so that it does not change the temperature of or otherwise damage the tissue. This jar or container containing the tissue and antibiotics is then sealed or closed and placed on a rocker platform and agitated for, preferably, between 60 and 90 minutes. Such rocking or agitation of the tissue within the antibiotic solution further cleans the placental tissue components of contaminants and bacteria.

Following agitation of the umbilical cord tissue in the antibiotic solution, the cord tissue is then stored in antibiotic solution and transferred to a refrigerator at a temperature of 1-10 °C for a minimum of four days up to a maximum of fifteen days.

After the umbilical cord tissue has been stored for four to fifteen days in antibiotic solution, it can be removed from the refrigerator. Using sterile forceps, the umbilical cord tissue is gently removed from the jar or container and placed in a sterile basin containing sterile water or normal saline (0.9% saline solution). The umbilical cord tissue is allowed to soak in place in the sterile water/normal saline solution for at least 10 to 15 minutes. The umbilical cord tissue is then transferred to a Nalgene container filled with sterile water or normal saline (0.9% saline solution). The Nalgene container filled with water and umbilical tissue is then transferred to a shaker platform where it is agitated for 30-60 minutes. Once the agitation step has been completed, the umbilical cord tissue is then laid out over a fixture and prepared for lyophilization.

### Lyophilization Step

Preferably, the umbilical cord tissue is placed in an individual, sealed Tyvek pouch (or other commercially available pouch) and placed into a commercially available freeze drying chamber. Any lyophilization process known to one skilled in the art may be used, so long as the umbilical cord tissue is substantially dehydrated when the lyophilization process has been completed.

Other methods may be used to adequately dehydrate the umbilical cord tissue. Such techniques may include, but are not limited to chemical dehydration, or placing the tissue in a low humidity/high temperature environment for an adequate period of time until optimal dehydration of the umbilical cord tissue has been achieved. Such dehydration techniques are generally well-known to those having skill in the art.

### Applying Mesh Pattern to Dehydrated Umbilical Graft

The dehydrated umbilical cord tissue graft is subjected to a cutting process whereby the desired mesh pattern is applied to the graft. Several methods are available to administer the desired mesh pattern to the graft. The following cutting methods are examples, and are not exclusive. There are many methods of applying cut patterns to tissue grafts that are known to those having skill in the art. One method of applying the desired pattern to the graft involves the use of a die cutting system, such as the commercially available Biocut Systems® cutting die. The use of this system incorporates a custom designed die that is applied to the graft under pressure, so the that the pattern on the custom die is cut directly into the graft.

Another method of applying the desired pattern to the graft involves the use of a skin mesher, such as the commercial unit manufactured by 4Med, Ltd.® “Rosenberg” Adjustable Skin Mesher. The graft can be fed through the mesher, which can be adjusted to apply different cut patterns in several different mesh ratios. Once the desired mesh ratio is set, the graft is force fed through the mesher and the cutting pattern is applied as the graft exits the meshing teeth.

The mesh pattern may also be applied through the use of a commercially available laser cutting device such as the Optek Systems® laser cutting system. The schematics of the desired cut pattern can be programmed into the device, which will then use a high power laser light to cut the pattern into the graft as specified in the programmed schematics. The graft is held in place during the cutting process on a fixture that has been custom designed for this process. Laser cutting provides several advantages over other methods, including greater flexibility with respect to desired cutting patterns, particularly with closely-spaced cuts, and no dulling of cutting edges of mechanical cutting tools. Dulling of the mechanical cutting edges may result in incomplete cuts through the entire thickness of the graft, which may have a negative impact on graft quality and expansion capabilities, resulting in an inferior product.

In an embodiment, the cuts will be in a pattern which permits stretching or expansion of the graft at least 10-400% by applying a force that is less than its tensile strength.

In an embodiment, the cuts will be in a pattern which permits stretching or expansion of the graft at least 10-400% by application of a force that is less than the graft's tensile strength. By applying a force that is less than the graft's tensile strength, the graft will maintain the desired expanded configuration without compromising its structural integrity.

In an embodiment, the cuts will be in a pattern which permits stretching or expansion of the graft at least 10-400% without microtears forming in the graft. It is understood that “microtears” do not include the cuts intentionally made in the graft by a die, laser cutter, or other method.

In an embodiment, the cuts will be in a pattern which permits stretching or expansion of the graft at least 10-400% without tears visible to the naked eye forming from cuts in the graft. It is also understood that these tears do not include the cuts intentionally made in the graft by a die, laser cutter, or other method. In an embodiment, tears form from less than 10%, 9%, 8%, 7%, 6%, 5%, 4%, 3%, 2%, or 1% of the cuts.

In an embodiment, the cuts will be in a pattern which permits stretching or expansion of the graft at least 10-400% without strands of the graft breaking. In an embodiment, less than 10%, 9%, 8%, 7%, 6%, 5%, 4%, 3%, 2%, or 1% of the strands break.

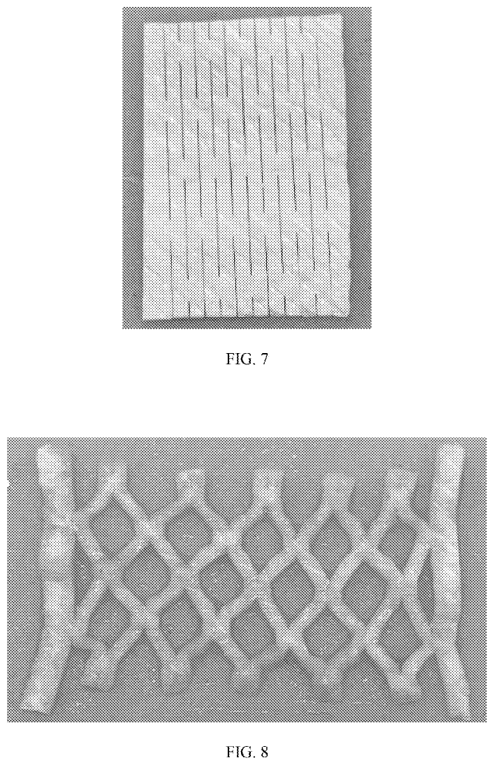

In an embodiment, the cuts will not be made within a certain distance from at least one edge of the graft, so as to produce at least one border for ease of handling, This is exemplified in FIGS. 7 and 8, which show a graft having at two borders, in unexpanded and expanded form, respectively. In an embodiment, the border is thicker than at least one strand created by the mesh pattern. In an embodiment, each border is, independently, 0.2 mm-50 mm thick.

In some embodiments, the cuts may comprise parallel, staggered cuts, as exemplified in FIG. 1. In some embodiments, the cuts comprise two distinct portions of cuts, wherein each portion comprises closely-spaced pairs of parallel cuts, and wherein the first portion's cuts and the second portion's cuts are at an angle with respect to each other, as exemplified in FIG. 2. In some embodiments, the cuts are arranged so that the expanded graft will have a “spiderweb” pattern, as exemplified in FIG. 3. In some embodiments, the cuts are arranged in repeating patterns of straight and slanted slits, as exemplified in FIGS. 4-6. For example, FIGS. 4-6 show grafts prepared with staggered, repeating units, each repeating unit having a central, longer slit, on either side of which are two angled slits which meet perpendicular to the center of the central slit, so as to form a obtusely-angled V-shape.

### Packaging

After the desired mesh pattern has been applied to the dehydrated umbilical cord tissue graft, the graft is then placed within a pouch. The graft may be placed into the pouch in the presence of ambient, atmospheric air, or it can be filled with an inert gas such as nitrogen, meant to displace the ambient, atmospheric air. This pouch is then sealed and placed within another pouch, which is also sealed once the inner pouch has been introduced. The inner pouch is traditionally referred to as the sterile pouch, while the outer pouch is considered non-sterile.

### Sterilization

The inner and outer pouch along with the resulting dehydrated umbilical cord tissue grafts are subjected to a terminal sterilization step. Terminal sterilization is accomplished by exposing the dehydrated umbilical cord tissue grafts to high energy, penetrating, ionizing radiation such as electron beam or gamma irradiation while the product is in its final packaging unit.

### Reconstitution of Meshed, Dehydrated Umbilical Graft

In order to administer the meshed umbilical cord graft to a subject, the end user must first reconstitute the graft by rehydrating it. Optimally, the rehydrating agent is 0.9% saline solution, but any suitable excipient may be used.

### Administration of Meshed, Dehydrated Umbilical Graft to a Subject

Once the umbilical cord graft has been reconstituted with the desired rehydrating agent, it is then applied to the wound site. Also, the graft may be hydrated in the wound site with the rehydrating agent or blood present from wound bed preparation. The reconstituted umbilical cord graft is expanded in order to achieve maximum coverage of the wound bed. The graft is expanded along a single axis by applying a force that is less than the graft's tensile strength, because application of force in excess of the tensile strength would tend to disrupt the structural integrity of the graft and cause it to rupture. One of ordinary skill in the art will understand that structural integrity is required in order for the graft to be easily applied to a site, and, in some embodiments, may be evaluated by tensile strength, tears, or strand breaks, as discussed hereinabove.

### Experimental

Grafts were prepared having a number of different cut patterns and expanded, in order to determine the suitability of the patterns for expandable grafts.

### Experiment 1: Slit Design (FIG. 1)

The slit prototype is very similar in pattern to the slits achieved by a traditional skin graft mesher, but the slit lengths and spacing were optimized to achieve peak expansion. The expanded graft had 1 mm thick strands of tissue surrounding large holes where incisions were made. The graft maintained excellent integrity during product handling and suturing. Both laser and die cut methods of manufacturing were successful at creating complete cuts. Expanded grafts were measured at an average of 3.1 times the original size of the graft.

### Experiment 2: Zig Zag Design (FIG. 2)

The zig zag incision pattern, when expanded, creates small holes with strips of tissue covering each small hole. This design allowed for better wound coverage because the tissue strands were diagonally positioned and very close together. The graft maintained excellent integrity during product handling and suturing. The laser cut method was successful as manufacturing these incisions. Because the distance between slits is very small, the die could not be manufactured to specification, so the die cut method was not possible. Expanded grafts were measured at an average of 2.2 times the original size of the graft.

### Experiment 3: Spiderweb Design (FIG. 3)

The spiderweb design was created by positioning the slit design at different angles from the center of the graft to create a hexagonal effect. This prototype did not expand. Product handling was mediocre as the corners of the graft detached easily. Suturability was not attempted due to the lack of expansion achieved and the prototype was no longer considered a viable option for the project.

### Experiment 4:“Evil Eye” Design (FIG. 4)

The evil eye design is a dense series of straight and slanted slits. This design allowed for excellent wound coverage and expansion, but the strands of tissue were very thin (less than 1 mm thick). The graft maintained excellent integrity but product handling was slightly difficult as the strands of tissue tangled easily. The product allowed for excellent suturability. The laser method was successful at manufacturing incisions. The die could not be manufactured to specification therefore the die cut method was not possible. Expanded grafts measured at an average of 3.3 times the original size of the graft.

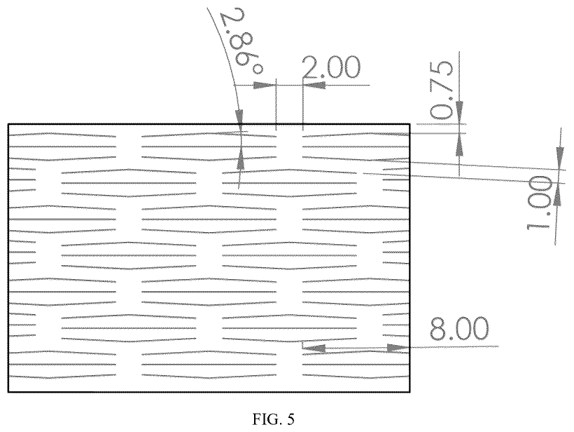

### Experiment 4.1: Evil Eye Design 2 (FIG. 5)

The evil eye design was redesigned to create thicker strands of tissue. For the 4.1 design, the number of slits was reduced significantly. Product handling and suturability were excellent due to thicker strands of tissue, but expansion reduced to an average of 1.8 times the original size of the graft.

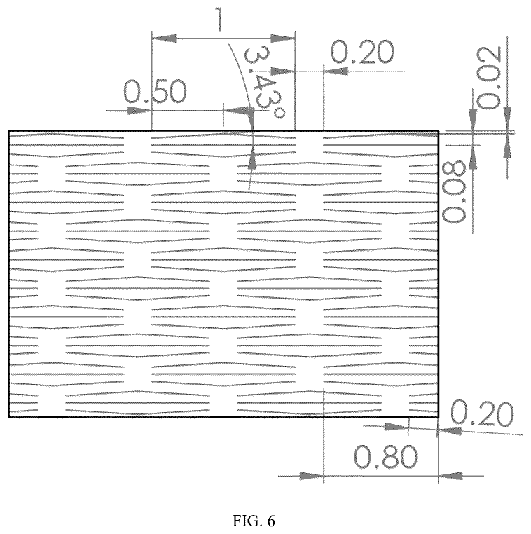

### Experiment 4.2: Evil Eye Design 3 (FIG. 6)

The evil eye design was redesigned to create thicker strands of tissue, but also allow for optimal expansion. For the 4.2 design, the number of slits was reduced slightly. Suturability was excellent due to the thicker strands of tissue, but product handling was still a little difficult due to the tangling of strands. The average expansion was 2.0 times the original size of the graft.

Experiment 5: Expansion of grafts

Photographs of an umbilical graft prepared according to Experiment 1 are shown, both before and after expansion (FIG. 7 and FIG. 8, respectively).

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

Citation or identification of any reference in this application is not an admission that such reference is available as prior art. The disclosures of all cited references including publications, patents, and patent applications are expressly incorporated herein by reference in their entirety.

Efforts have been made to ensure accuracy with respect to numbers used (e.g. amounts, temperature, etc.) but some experimental errors and deviations should be accounted for.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. One skilled in the art will recognize many methods and materials similar or equivalent to those described herein, which could be used in the practicing the subject matter described herein. The present disclosure is in no way limited to just the methods and materials described.

What is claimed is:

1. A meshed, expandable, and sterile umbilical tissue allograft.

2. The umbilical tissue allograft of claim 1, wherein said allograft comprises pre-determined cuts.

3. The umbilical tissue allograft of claim 2, wherein the cuts are in an engineered pattern.

4. The umbilical tissue allograft of claim 3, wherein all of said cuts are parallel to one another.

5. The umbilical tissue allograft of claim 4, wherein said cuts are 0.1-20 mm in length.

6. The umbilical tissue allograft of claim 4, wherein the distance between two adjacent cuts is between 0.1-10 mm.

7. The umbilical tissue allograft of claim 3, wherein a first portion of said cuts are parallel to one another, a second portion of said cuts are parallel to one another, and the first portion and the second portion are oriented at an angle of 1°-180° relative to each other.

8. The umbilical tissue allograft of claim 7, further comprising a third portion of cuts which is not parallel to the first portion or to the second portion.

9. The umbilical tissue allograft of claim 3, comprising no more than five portions of cuts, wherein each of said no more than five portions of cuts are oriented at an angle of 1°-180° relative to each of said other portions.

10. The umbilical tissue allograft of claim 2, wherein said cuts comprise no more than 20%, 15%, 10%, 9%, 8%, 7%, 6%, 5%, 4%, 3%, 2%, 1%, 0.5%, or 0.1% of the surface area of the allograft.

11. The umbilical tissue allograft of claim 10, wherein said cuts are slits.

12. The umbilical tissue allograft of claim 1, wherein the allograft may be expanded by at least 10%-400% along one axis by application of a force that is less than its tensile strength.

13. The umbilical tissue allograft of claim 1, wherein the allograft may be expanded so as to increase its surface area by at least 10%-400% by application of a force that is less than its tensile strength.

14. The umbilical tissue allograft of claim 1, wherein the umbilical tissue allograft is dehydrated.

15. The umbilical tissue allograft of claim 14, wherein the umbilical tissue allograft is lyophilized.

16. The umbilical tissue allograft of claim 1, contained within a sealed pouch.

17. The umbilical tissue allograft of claim 16, wherein said sealed pouch is deoxygenated.

18. A method of covering a wound comprising contacting a wound with a sterile umbilical tissue allograft of claim 1, wherein said contacting comprises expanding said sterile umbilical tissue allograft by at least 10%-400% along one axis to cover said wound.

19. A method of treating a subject in need thereof using the umbilical tissue allograft of claim 1, said method comprising
i) expanding said umbilical tissue allograft by at least 10%-400% along one axis, or expanding said umbilical tissue allograft so as to increase its surface area by at least 10%-400%; and
ii) applying the expanded umbilical tissue allograft to the subject.
